(19) United States
(12) Patent Application Publication
(10) Pub. No.: US 2002/0076833 A1
(43) Pub. Date: Jun. 20, 2002

(54) Analysis of biological samples utilizing a coated solid phase

(76) Inventors: Michael R. Henry, Grayslake, IL (US); David C. Leahy, Grayslake, IL (US); Ronald J. Shebuski, Bergland, MI (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/920,369

(22) Filed: Aug. 1, 2001

Related U.S. Application Data
(60) Provisional application No. 60/222,204, filed on Aug. 1, 2000.
(60) Provisional application No. 60/299,129, filed on Jun. 18, 2001.

(51) Int. Cl. G01N 33/543

(52) U.S. Cl. 436/518

(57) ABSTRACT

The invention disclosed herein comprises methods for analyzing a biological sample, such as undiluted or diluted whole blood, as well as fractions thereof, for the presence or absence and/or the concentration of disease-specific and/or other medical condition-specific markers. Such markers may include platelet activation and coagulation activation markers. The methods may comprise combining the biological sample with a coated solid phase and analyzing for the presence or absence and/or the concentration of the markers. The analysis may be performed either before or after separation of the solid phase from the biological sample. The analysis may be performed on the combined components or on any of the separated components. A preferred solid phase may be paramagnetic microparticles coated with antibodies or proteins specific for platelet activation and/or coagulation activation markers.

### CROSS REFERENCE TO RELATED APPLICATIONS

This application claims priority from U.S. Provisional Patent Application No. 60/222,204, filed Aug. 1, 2000 (Atty. Docket No. MBHB00-555) and U.S. Provisional Patent Application No. 60/299,129, filed Jun. 18, 2001 (Atty. Docket No. MBHB00-555-A). All patents, patent applications (published or unpublished) and other scientific or technical writings referred to herein are hereby incorporated by reference to the extent that they are not contradictory.

### BACKGOUND OF THE INVENTION

1. Field of the Invention

The invention is related to the field of blood analysis. In particular, blood analysis using a solid phase coated with marker-specific compounds. More in particular, blood analysis wherein the solid phase comprises paramagnetic particles and the marker-specific compounds comprise antibodies, receptors, ligands, proteins, peptides, cytokines, chemokines, small molecules and the like. Even more in particular, the invention is related to the analysis of activated and unactivated platelets from whole blood as well as the identification of chemical markers associated with the coagulation process.

2. Description of Related Art

Separation of platelets from whole blood is generally accomplished by centrifugation of the blood at 150×g for 10 min. The platelet-rich plasma (PRP) fraction is then carefully removed from the top layer and the platelets are subsequently isolated for later use such as transfusion and/or diagnostic tests of platelet function. However, platelets are very prone to artifactual activation when they are centrifuged and handled (Metcalfe, P., Williamson, L. M., Reutelingsperger, C. P., Swann, I., Ouwechand, W. H. & Goodall, A. H. (1997) “Activation during preparation of therapeutic platelets affects deterioration during storage: a comparative flow cytometric study of different production methods.” Br. J. Haemotol., vol. 98, no. 1, pp. 86-95). Artifactual activation has hampered the platelet diagnostic field for decades as the platelet releases many substances upon activation that could be utilized as new diagnostic markers in the identification of patients with “hyperactive platelets.” However, often platelet specific markers, such as beta-thromboglobulin (Mundal, H. H., Hjemdahl, P., Urdal, P., Kierulf, P., Perneby, C., Bergt, K. & Gjesdal, K. (1998) “Beta-thromboglobulin in urine and plasma: influence of coronary risk factors.” Thromb. Res., vol. 90, no. 5, pp. 229-237) and thromboxane B-2 (Ciabattoni, G., Maclouf, J., Catella, F., FitzGerald, G. A. & Patrono, C. (1987) “Radioimmunoassay of 11-dehydrothromboxane B2 in human plasma and urine.” Biochim. Biophys. Acta, vol. 918, no. 3, pp. 293-297) are increased as a consequence of isolating the platelets for assay by centrifugation and further handling in the assay. Thus, these markers have not been adapted in the identification of platelet activation as initially hoped. It is an object of this invention to provide a novel method for separating platelets from whole blood, without centrifugation, such that subsequent analysis of platelet specific markers can be accomplished without artifactual elevation of such markers due to processing.

Platelet activation and subsequent aggregation are known to play a pivotal role in the acute pathophysiology of thrombus formation, stroke and acute coronary syndromes (ACS). ACS patients experiencing unstable angina and/or non Q-wave myocardial infarction are prone to plaque rupture and thrombus formation which is amenable to a host of pharmacological agents (thrombolytics, GPIIb/IIIa antagonists, anti-coagulants). Thus, it is imperative that platelet activation be assessed rapidly and accurately such that appropriate therapeutic interventions be employed to salvage ischemic myocardium that is at risk of infarction (death). Methods that allow for separation of platelets from whole blood without centrifugation, will be valuable in developing new assays for detecting “hyperactive” platelets that may contribute to disease states.

All platelets, either unactivated, activated or circulating as microparticles, express glycoprotein 1b (GP1b) on their surface (White, J. G., Krumwiede, M. D. & Escolar, G. (1999) “Glycoprotein 1b is homogeneously distributed on external and internal membranes of resting platelets.” Am. J. Pathol., vol. 155, no. 6, pp. 2127-2134). Thus, GP1b represents an appropriate target for identifying all circulating platelets and platelet microparticles. In the present invention, anti-GP1b monoclonal antibodies may be employed to “capture” platelets in which the antibody is coated onto the surface of paramagnetic particles. Mixing whole human blood with GP1b-coated paramagnetic particles, followed by magnetic separation, results in platelet capture and substantial depletion of platelets from a given sample.

### BRIEF SUMMARY OF THE INVENTION

This invention relates to the analysis of biological samples using one or more coated phase(s). The biological sample may be, but is not limited to, undiluted and/or diluted whole blood, undiluted and/or diluted blood plasma, as well as given fraction(s) of fractionated whole blood. The solid phase may be, but is not limited to, paramagnetic particles. The solid phase may be coated with one or more marker-specific protein tracers including, but not limited to, antibodies, receptors, ligands, proteins, peptides, cytokines, chemokines, small molecules and the like.

This invention further relates to methods for separating platelets without activating the platelets during the separation process to provide a platelet sample containing activated and unactivated platelets along with platelet-derived microparticles wherein the activated platelets are activated by physiological processes in vivo and not by the separation process.

The invention includes separated platelet compositions that are substantially unactivated by the separation process and comprising both physiologically activated platelets and unactivated platelets along with platelet-derived microparticles. The invention includes assays of the separated platelet samples based on the markers from the physiologically activated platelets.

A preferred method for separating platelets and platelet derived microparticles involves attaching an antibody or protein that specifically binds to platelets and platelet derived microparticles onto paramagnetic particles and contacting a diluted or undiluted whole blood sample or fraction thereof with the antibody-coated paramagnetic particles, magnetically separating the paramagnetic particles and attached platelets and removing the remaining substantially platelet free supernatant from the paramagnetic particles-platelet complexes. This separation provides a platelet composition where platelets are not activated by the separation process and the only activated platelets in the composition are those that have been physiologically activated in vivo. Thus, analysis of cellular markers in this composition is a more accurate measurement of the in vivo physiological activated platelets.

This invention also relates to methods for separating platelets and platelet-derived microparticles without activating the platelets during the separation process to provide a sample substantially free of platelets and platelet-derived microparticles.

The invention includes assays of the substantially platelet-free samples based on markers which could be significantly influenced by the presence of physiologically-activated platelets within the sample.

A preferred method for obtaining a substantially platelet free sample involves separating platelets by attaching an antibody that specifically binds to platelets onto paramagnetic particles and contacting a whole blood sample or diluted whole blood sample with the antibody-coated paramagnetic particles, magnetically separating the paramagnetic particles and attached platelets and separating the remaining platelet-free supernatant from the paramagnetic particle/platelet complexes. This separation process provides a sample composition substantially free of platelets and physiologically unaltered by the separation process. Thus, analysis of soluble markers in this composition is a more accurate measurement of the in vivo physiological state.

It is also contemplated that this invention would be applicable to the identification of other diseases and conditions. By judicious selection of the material coating the paramagnetic particles (e.g., including, but not limited to, antibodies (polyclonal and/or monoclonal), ligands, receptors, proteins, peptides, cytokines, chemokines, small molecules and the like), one would be able to analyze undiluted and/or diluted whole blood, and/or any fraction thereof, for the presence or absence of markers of these other diseases and conditions. These other diseases and conditions, and representative markers, include, but are not limited to, the following:

Atherosclerosis/Inflammation/Arthritis

Proteins: VCAM-1, ICAM-1, P- and E-selectin, P-selectin glycoprotein ligand-1 (PSGL-1), PECAM-1, C-reactive protein (hCRP), ox-LDL, HDL, LDL, Apolipoprotein A1 (Apo A-1), total cholesterol, LP(a), CD15, CD40, Interleukin-6 (IL-6), Interleukin-1 receptor antagonist (IL-1ra ), Tumor Necrosis Factor (TNF), Tissue Factor (TF), Tissue Factor Pathway Inhibitor (TFPI), Complement C3a and C5a, C3 and C5 Convertase, Factor D, Kallikrein, Plasmin, C1-Inhibitor, soluble CR1, etc.

Chemokines: Monocyte Chemoattractant Protein-1 (MCP-1), MCP-4, Regulation on Activation Normal T-cell Expressed and Secreted (RANTES), Interleukin-8 (IL-8), Stromal Cell Derived Factor-1 (SDF-1), etc.

Diabetes

Proteins: P- and E-selectin, GPIIb/IIIa, lysosomal GP53, thrombospondin, Glucose associated Hemoglobin (GHb), glycohemoglobin A(1c), gamma globulin, Insulin-like growth factor (IGF-1), Insulin-like Growth Factor Binding Proteins 1-6, Pregnancy Associated Plasma Protein A (PAPP-A), Receptor for Advanced Glycation End-Products (RAGE), etc.

Transplant Rejection

Proteins: hCRP, E- and L-selectin, CD18, CD11a, CD11b, Interleukins (IL-1, II-2, IL-6, II-15), Complement C3a and C5a, C3 and C5 Convertase, Factor D, soluble CR1, Interferon gamma (IFN-gamma), Chemokines (CCR2, CCR3, CCR5), TNF alpha, IgG, etc.

Alzheimer's Disease/Vascular Dementia

Proteins: Beta-amyloid peptide, Protein tau, Beta-amyloid precursor protein, Abeta1-40, Abeta1-42, presenillins, ApoE4, C3a, C5a, C3 and C5 Convertase, ERp57, etc.

Additional Thrombosis/Stroke/Peripheral Arterial Disease Targets (Other than those Already Mentioned (i.e., GPIIb/IIIa, P-selectin, D-dimer, PTF1.2))

Proteins: Factor (FXa), Factor VIIa (FVIIa), Factor IXa (FIXa), Factor V(FV), Thrombin (FIIa), Factor XIIIa (FXIIIa), Tissue Factor (TF), Tissue Factor Pathway Inhibitor (TFPI), CD45, activated Partial Thromboplastion Time (aPTT), Prothrombin Time (PT/INR), Thrombomodulin, Thrombospondin, Thromboxane (TxA-2), Plasminogen Activator Inhibitor-1 (PAI-1), Thrombin Activatable Fibrinolysis Inhibitor (TAFI), Tyrosine Kinase SYK, Angiotensin IV, P2T, von Willebrand's Factor (vWF), Fiobrinopetide A (FPA), Fibrinopeptide B (FPB), Fibrin Degradation Products (FDP's), Thrombin-Antithrombin Complex (TAT), Pentraxin (PTX3), t-PA (tissue plasminogen activator), u-PA (urokinase plasminogen activator), Plasminogen, Plasmin, Factor XIII, alpha-2-plasmin inhibitor, alpha-1 anti-trypsin inhibitor, etc.

Hemophilia and Related Disorders

Proteins: Factor VIII, Factor IX, etc.

Others

Proteins: peptides and proteins generated during platelet activation and coagulation, neurological and neuro-associated peptides and proteins, antibodies to heparin in heparin associated thrombocytopenia, etc.

### BRIEF DESCRIPTION OF THE SEVERAL VIEWS OF THE DRAWINGS

FIG. 1 shows a graph illustrating correlation of platelet counts of whole blood diluted in Cellpack diluent and in phosphate buffered saline (PBS), pH 7.4 supplemented with 5% bovine serum albumin (BSA) and 10% CTAD.

FIG. 2 shows a graph illustrating a linear response of platelet counts in diluted whole blood.

FIG. 3 shows the percent platelet capture versus capture time in diluted whole blood. The capture data for three concentrations of coated particles is displayed.

FIG. 4 shows a correlation of fluorescence intensity versus soluble P-selectin (at various concentrations in diluted plasma) captured using anti-P-selectin (anti-CD62P)-coated paramagnetic microparticles.

FIG. 5 shows a correlation of fluorescence intensity versus membrane P-Selectin for different sample preparation conditions using a 1-step assay format.

FIG. 6 shows a correlation of fluorescence intensity versus membrane P-Selectin for different sample preparation conditions using a 2-step assay format.

FIG. 7 shows a correlation of fluorescence intensity versus membrane GPIIb/IIIa for different sample preparation conditions (ie., sample volume).

FIG. 8 shows a correlation of fluorescence intensity versus membrane P-Selectin for different sample preparation conditions (i.e., sample volume).

### DETAILED DESCRIPTION OF THE INVENTION

The term “label” means a group or compound attached to an antibody or an analyte or an analyte analogue that renders the reaction between the antibody or analyte or analyte analogue detectable. Representative examples of labels include enzymes, radioactive elements, fluorophores, and chemicals that produce light. A label is any substance, either alone or in conjunction with other substances, that can be attached to an appropriate molecule and that is capable of producing a signal that is detectable by visual or instrument means. Various labels include catalysts, enzymes, liposomes, and other vesicles containing signal producing substances such as chromogens, catalysts, fluorescent compounds, chemiluminescent compounds, enzymes, radioactive elements and the like. In this invention, the preferred label is fluorescent. The term “tracer” is synonymous with the term “label”.

The term “solid phase” means a plurality of microparticles having specific binding members chemically or physically bound thereto. Other solid phases that are known to those skilled in the art include the walls of wells or reaction trays, tubes, polymeric beads, nitrocellulose strips, membranes, chromatographic columns and the like. A preferred solid phase comprises microparticles made of polystyrene containing a layer of iron oxide rendering them paramagnetic. The preferred method of separating the particles from the test sample involves capture of the particles by means of a magnetic field. In this invention the preferred method, the solid phase consists of paramagnetic microparticles having specific binding members chemically or physically bound thereto.

The term “sample”, “biological sample”, and the like mean a material suspected of containing an analyte. The sample can be used directly as obtained from the source or following a pretreatment to modify the character of the sample. The sample can be derived from whole blood. The sample can be treated prior to use, such as preparing plasma from whole blood, diluting viscous fluids, and the like. Methods of treatment can include fractionation, filtration, extraction, concentration, the addition of reagents and the like.

The following non-limiting examples will further explain the invention

### EXAMPLE 1

### Platelet Capture

This example illustrates the capture and removal of platelets from whole blood or diluted whole blood.

Materials and Methods

Whole blood was obtained from healthy volunteers and collected into centrifuge tubes containing citrate theophylline adenosine dipyridamole (CTAD), pH 5.4 anticoagulant at a 9:1 ratio. A volume of whole blood was diluted to 1.85% in phosphate buffered saline (PBS), pH 7.4, supplemented with 5% bovine serum albumin (BSA) and 10% CTAD.

Platelet cell counts were examined by means of an automatic analyzer (Sysmex Microcellcounter F-800 Hematology Analyzer, Sysmex Corp. of America, Long Grove, Ill.; see also Fujimoto, K. (1999) “Principles of Measurement in Hematology Analyzers Manufactured by Sysmex Corporation” Sysmex Journal International, vol. 9, no. 1, pp. 31-44). Each sample condition was pipetted into a Sysmex Disposable Sample Beaker (DB-1) and further diluted with 10.0 mL of Cellpack Whole Blood Diluent ( CPK-310 A) and assayed in duplicate.

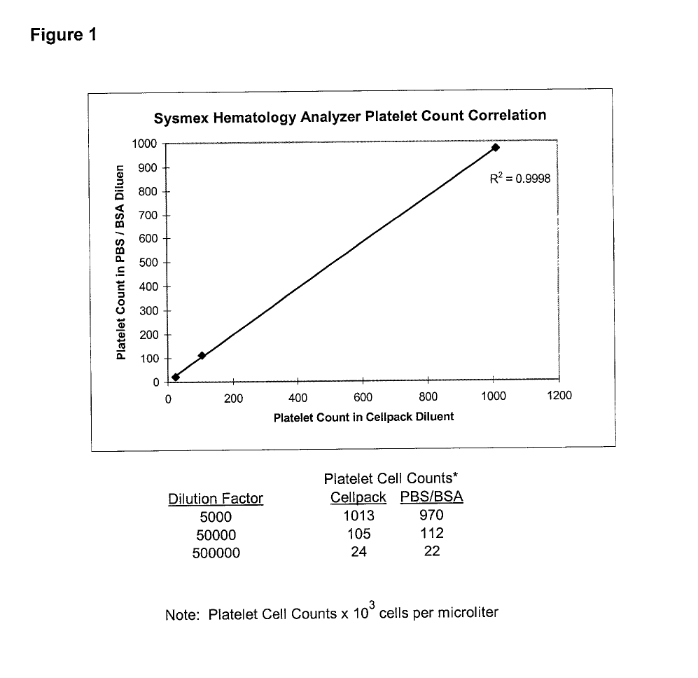

Platelet counts from whole blood were compared as a function of dilution with either Cellpack (buffer supplied with the Sysmex Microcellcounter F-800 Hematology Analyzer system) or with phosphate-buffered saline supplemented with 5% bovine serum albumin (PBS/BSA) diluent. The data shown in FIG. 1 demonstrates that platelet counts decreased as a function of whole blood dilution and platelet counts obtained from (PBS/BSA) diluent correlated well with the Sysmex Hematology Analyzer Cellpack diluent.

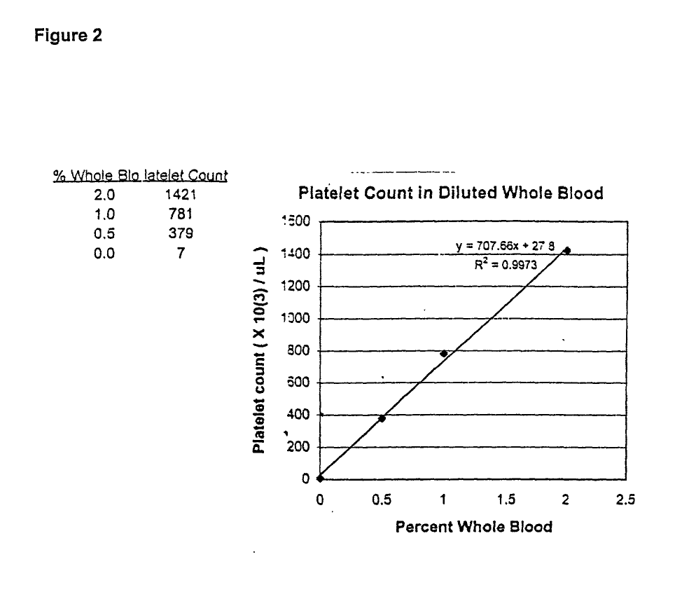

In order to demonstrate measurements were performed within the linear detection portion of the Sysmex Microcellcounter F-800 Hematology Analyzer, the following experiments were conducted: 20 uL of whole blood was pipetted into 980 uL of PBS containing 5% BSA, 10% CTAD and then serially dilute as appropriate. 100 uL of prediluted sample were deposited into a DB-1 Sample Beaker and further diluted with 10.0 mL of Cellpack Whole Blood Diluent for Platelet cell count measurements. The data shown in FIG. 2 demonstrates that platelet counts in 1.85%-diluted were performed within the linear portion of the Sysmex platelet detection curve. Plotted is a dilution curve from 0-2% with a corresponding high correlation coefficient (0.9973).

3.7 micron (pm) carboxyl-modified paramagnetic microparticles were coated with murine monoclonal antibody (mAb) which specifically recognizes the platelet membrane surface component glycoprotein GP1b, (CD42b, Biodesign, N42409M, lot 6G1996) and were re-suspended in phosphate buffered saline (PBS)-supplemented with 1% bovine serum albumin (BSA) buffer at 7.4% w/v. 50 μL of the paramagnetic microparticle preparation was added to a reaction tube, separated magnetically and supernatant removed. The particles were resuspended with 100 μL of 1.85% whole blood dilution and incubated at room temperature for 30 minutes. The paramagnetic particles were separated magnetically and supernatant removed for platelet cell count analysis.

Table 1 represents the results of this experiment. Whole human blood was diluted to 1.85% and separation of platelets performed with murine monoclonal antibody anti-GP1b coated paramagnetic microparticles. The samples were incubated for 30 minutes at room temperature and then the microparticles were separated from the solution magnetically and the supernatant removed. The platelet counts from each reaction supernatant was pipetted into a disposable sample beaker (DB-1) and further diluted with 10 ml of Cellpack diluent and platelet counts determined in the Hematology Analyzer.

Incubation of whole blood with anti-GP1b murine monoclonal antibody coated paramagnetic particles resulted in approximately 90% capture of platelets in the sample. These results demonstrate that paramagnetic microparticles, coated with a murine monoclonal antibody directed against platelet GP1b, followed by magnetic separation, are capable of capturing platelets from human whole blood with no other traditional manipulations such as centrifugation for subsequent use of the platelet fraction in determining the state of platelet activation in patients.

Following the above methods, monoclonal antibodies to antigens listed below and magnetic particles coated with such antibodies are used to separate platelets without activation:

CD42a (GPIX)—part of GP1b-IX-V complex;

CD42b (GP1b-alpha)—145 kD platelet binding site for vWF and thrombin;

CD42c (GP1b-beta)—25 kD disulfide bonded to alpha subunit;

CD42d (GPV);

CD41 (GPIIb, also known as alpha IIB integrin);

CD61 (GPIIIa)—beta 3 subunit of GPIIb/IIIa complex (alpha 2b, beta 3);

CD41/CD61 (GPIIb/IIIa complex)—receptor for fibrinogen, fibronectin, von Willebrand factor, and other adhesion proteins containing the Arg-Gly-Asp motif

CD36 (GPIV)—platelets/monocytes;

CD49b (VLA-2)—platelets/monocytes;

CD51 (alpha V, beta 3)—vitronectin receptor;

CD62p (P-selectin)—platelets; and

CD107a (LAMP-2)—lysosomal protein translocated to cell surface after activation

CD41a (GPIIb/IIIa)—intact IIb/IIIa complex; fibrinogen, von Willebrand factor, fibronectin and vitronectin receptor.

TABLE 1
Platelet Capture in Whole Human Blood

| | | | Avg. - |
|---|---|---|---|
| Results: | | Avg.* | bkgd.* |
| Test 1 | | | |
| No Particle Control | 1334, 1302 | 1318 | 1309 |
| Positive Control 1** | 89, 104 | 95 | 86 |
| Positive Control 2** | 87, 101 | | |
| Sysmex System Buffer | 10, 8 | 9 | |
| Percent Capture | 93.4% Capture | | |
| Test 2 | | | |
| No Particle Control | 1121, 1086, | 1111.3 | 1093.3 |
| No Particle Control | 1102, 1136 | | |
| Positive Control 1** | 163, 159 | 139.8 | 121.8 |
| Positive Control 2** | 116, 121 | | |
| Sysmex System Buffer | 17, 19 | 18.0 | |
| Percent Capture | 88.9% Capture | | |
| Test 3 | | | |
| No Particle Control | 1142, 1173 | 1144.5 | 1126.5 |
| No Particle Control | 1130, 1133 | | |
| Positive Control 1** | 30, 72 | 63.0 | 45.0 |
| Positive Control 2** | 73, 77 | | |
| Sysmex System Buffer | 17, 19 | 18.0 | |
| Percent Capture | 96.0% Capture | | |

| Summary of Tests 1, 2 & 3 | |
|---|---|
| (n = 3 human subjects) | % Capture |
| Test 1 | 93.4 |
| Test 2 | 88.9 |
| Test 3 | 96.0 |
| Mean ± SEM | 92.8 ± 3.6% |

*(cell count × 10³ cells/μL)
**positive control (anti-GP1b paramagnetic particles)

### EXAMPLE 2

### Platelet Capture in Diluted Whole Blood

This example illustrates the dose response of time and solid phase percentage (surface area) to percent platelet capture from whole blood samples.

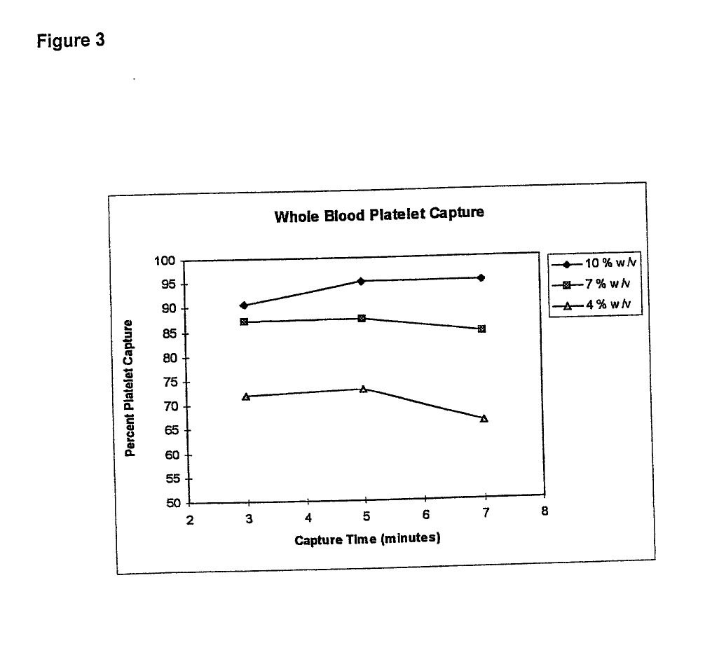

A multivariant designed experiment was used to examine platelet capture in diluted whole blood. Experimental parameters were: incubation time was about 3-7 minutes; antibody coating concentration was about 6-50 pg; particle concentration was about 4-10% (w/v). The assay was carried out as follows: whole blood was diluted to 2% with phosphate buffered saline (PBS) at pH 7.2 supplemented with 1% bovine serum albumin (BSA). 100 μL of paramagnetic anti-GP1b-coated particles were added to 100 μL of diluted whole blood at room temperature. Following an incubation period, the particles were separated magnetically and the supernatant was removed for testing in a Sysmex F-800 Hematology Analyzer Microcellcounter as previously described in example 1. The platelet concentration per μL was determined for each condition and compared to the original 2% stock solution. Particle coating concentration, particle % (w/v) and incubation times were varied. The results of these experiments are displayed in FIG. 3. The data demonstrates that effective platelet removal from whole blood is effected significantly by the percentage of solid phase utilized. Through the addition of increased higher percent solid phase concentrations, in turn ever increasing percentage of platelets are removed from the sample. The results indicate that for any given whole blood sample or whole blood sample dilution effective platelet capture is achievable if sufficient time, antibody coating concentration and percentage of anti-platelet coated solid phase is utilized.

### EXAMPLE 3

This example further illustrates the capture and removal of platelets from whole blood or diluted whole blood.

Whole blood was obtained from healthy volunteers and collected into centrifuge tubes containing D-phe-pro-arg-chloromethylketone (PPACK, dihydrochloride) anticoagulant. A volume of whole blood was diluted to 2.0% in phosphate buffered saline (PBS), pH 7.4, supplemented with 1% bovine serum albumin (BSA)

100 uL of Murine monoclonal antibody anti-GP1b coated paramagnetic microparticles were added to 100 μL of a 2.0% whole blood dilution and incubated at room temperature for 5 minutes. The paramagnetic particles were separated magnetically and the supernatant removed for platelet cell count analysis as previously described in Example 1 with a Sysmex Microcellcounter F-800 Hematology Analyzer.

TABLE 2

| | | | Avg. - |
|---|---|---|---|
| | Avg.* | bkgd.* | |
| No Particle Control | 601, 605 | 601 | 599 |
| No Particle Control | 594, 605 | | |
| Positive Control 1** | 67, 68 | 59 | 57 |
| Positive Control 2** | 52, 55 | | |
| Positive Control 3** | 53, 58 | | |
| Sysmex System Buffer | 2, 4, 3, 1, 1 | 2 | |
| Percent Capture | 90.5% Capture | | |

*(cell count × 10³ cells/μL)
**positive control (anti-GP1b paramagnetic particles)

### EXAMPLE 4

### Soluble P-selectin Assay in Plasma

This example illustrates the feasibility of assaying substantially platelet free samples for markers which can be influenced by the presence of physiologically activated platelets within the sample. In this example soluble P-selectin concentrations are measured in substantially platelet free samples to allow for discrimination from membrane P-selectin.

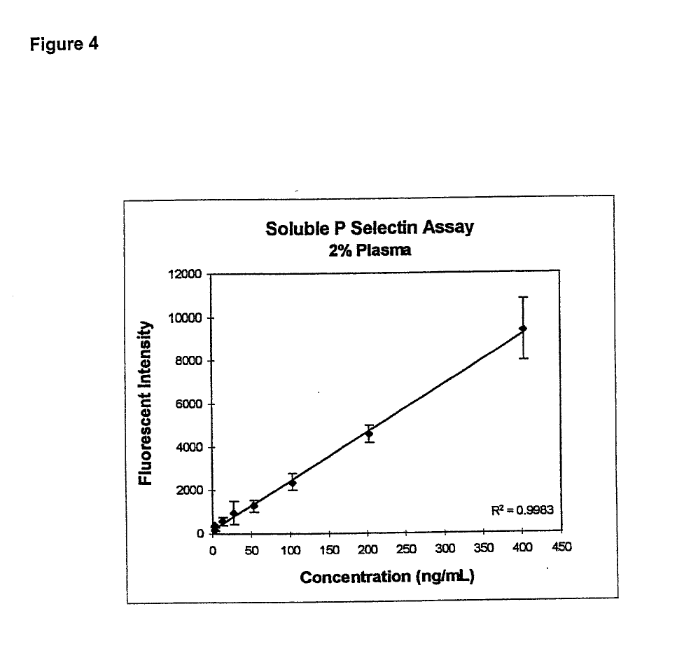

Recombinant human P-selectin was added to 2.0% platelet-free human plasma sample at the following concentrations: 400, 200, 100, 50, 25, 10 and 1.0 ng/mL. 50 μL of 0.125% (w/v) paramagnetic microparticles (1.5 micron (μm)) coated with an anti-P-selectin murine monoclonal antibody (Mab) (anti-CD62P) were added to 100 μL of each of the aforementioned sample concentrations of recombinant P-selectin in plasma at room temperature and incubated for 10 minutes The microparticles were separated magnetically and the supernatant was removed. The microparticles were washed by re-suspension with 200 μL of PBS supplemented with 0.5% (BGG) diluent. The microparticles were separated magnetically and the supernatant was removed. The washed microparticles were re-suspended in 50 μL of a 20 μg/mL solution of FITC-labeled anti-P-selectin (anti-CD62P) rabbit polyclonal antibody and incubated at room temperature for 10 minutes. The microparticles were separated magnetically and the supernatant removed. The microparticles were washed by re-suspension with 200 μL of (PBS) supplemented with 0.5% BGG diluent. The wash sequence was repeated and the particles were re-suspended in 0.1% SDS at pH 11. The microparticles were separated magnetically and the supernatants were transferred to a 96 well microtiter-plate and examined for fluorescence intensity. The graph in FIG. 4 illustrates how the incubation of samples with paramagnetic microparticles coated with anti-P-selectin murine monoclonal antibody followed by the addition of FITC labeled anti-P-selectin rabbit polyclonal antibody enables the detection of and quantitation of soluble P-selectin.

### EXAMPLE 5

### 1-Step Assay

Whole blood was obtained from a healthy volunteer and collected into a blood collection tube containing D-phe-pro-arg-chloromethylketone (PPACK, Dihydrochloride). An aliquot of whole blood was stimulated with 10 μM adenosine diphosphate (ADP) for two minutes and then fixed for two minutes with neutral buffered formalin (1% final concentration). Another aliquot was not treated with ADP, but only fixed with 1% neutral buffered formalin for two minutes

To 100 μL of each of the above three sample preparations the following reagents were added: 50 μL of FITC-labeled anti-P-selectin (anti-CD62P) rabbit polyclonal antibody (at 20 μg/mL) and 50 μL of 0.5% (w/v) paramagnetic microparticles (3.7 micron (μm)) coated with anti-glycoprotein GPIb (CD42b) murine monoclonal antibody (Mab). The mixtures were incubated at room temperature for either 10 or 15 minutes. The microparticles were separated magnetically and the supernatant was removed. The microparticles were washed by re-suspension in 200 μL of PBS supplemented with 1% bovine serum albumin (BSA) followed by magnetic separation of the microparticles and removal of the supernatant. This sequence was repeated and the microparticles were re-suspended in 0.1% SDS at pH 11. The microparticles were separated magnetically and the supernatants were transferred to a 96 well plate and examined for fluorescence intensity.

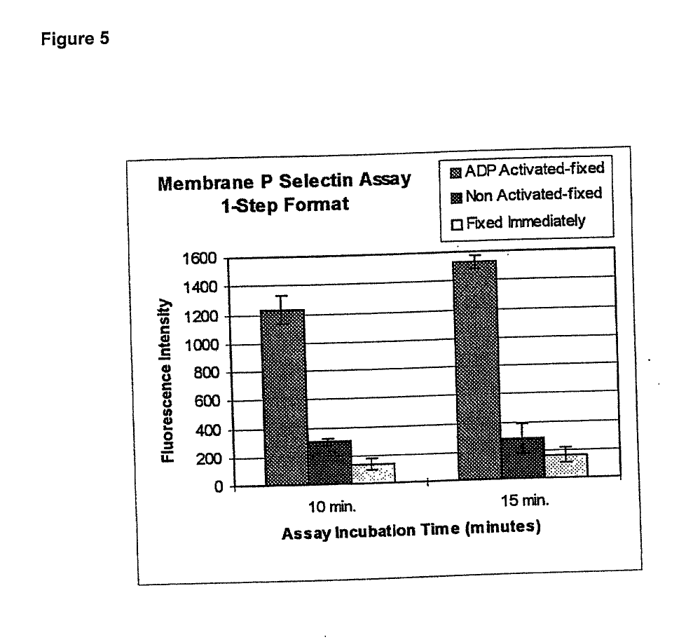

The data in table 3 as well as the graph in FIG. 5 illustrates how the incubation of whole blood with both paramagnetic microparticles coated with an anti-GP1b murine monoclonal antibody and a labeled anti-P-selectin rabbit polyclonal antibody enables the detection and discrimination of platelets that are expressing membrane-bound P-selectin from those that are not expressing membrane-bound P-selectin.

TABLE 3
FITC Signal Detected vs. Sample Preparation Conditions
(at representative incubation times)

| Sample Preparation Conditions | 10 min. | 15 min. |
|---|---|---|
| ADP agonist treated whole blood | 1235 | 1526 |
| Non agonist treated whole blood | 304 | 282 |

### EXAMPLE 6

### 2-Step Assay

Whole blood was obtained from a healthy volunteer and collected into a sample collection tube containing D-Phe-Pro-Arg-chloromethylketone (PPACK, dihydrochloride). An aliquot of whole blood was stimulated with 10 μM adenosine diphosphate (ADP) and 1.0 uM Epinepherine for two minutes and then treated for two minutes with neutral buffered formalin (1% final concentration). An additional aliquot of whole blood was stimulated with 10 μM ADP for two minutes and then treated for two minutes with neutral buffered formalin. An additional aliquot of whole blood was treated for two minutes with 1% neutral buffered formalin for two minutes without ADP stimulation.

50 μL of 0.5% (w/v) paramagnetic microparticles (3.7 micron (μm)), which were coated with anti-glycoprotein GP1b murine monoclonal antibodies (Mab), were added to 100 μL of each of the above sample preparations. The mixtures were incubated at room temperature for 10 minutes. The microparticles were separated magnetically and the supernatant was removed. The microparticles were washed by re-suspension in 200 μL of PBS supplemented with 1% BSA. The microparticles were again separated magnetically and the supernatant removed. The microparticles were re-suspended in 50 μL FITC-labeled anti-P-selectin (anti-CD62P) rabbit polyclonal antibody at 20 μg/mL. The assays were incubated at room temperature for an additional 10 minutes. The microparticles were separated magnetically and the supernatant removed. The microparticles were washed by re-suspension in 200 μL PBS supplemented with 1% BSA. The microparticles were again separated magnetically and the supernatant removed. The wash sequence was repeated and the microparticles re-suspended in 0.1% SDS at pH 11. The microparticles were separated magnetically and the supernatants transferred to a 96 well plate and examined for fluorescence intensity.

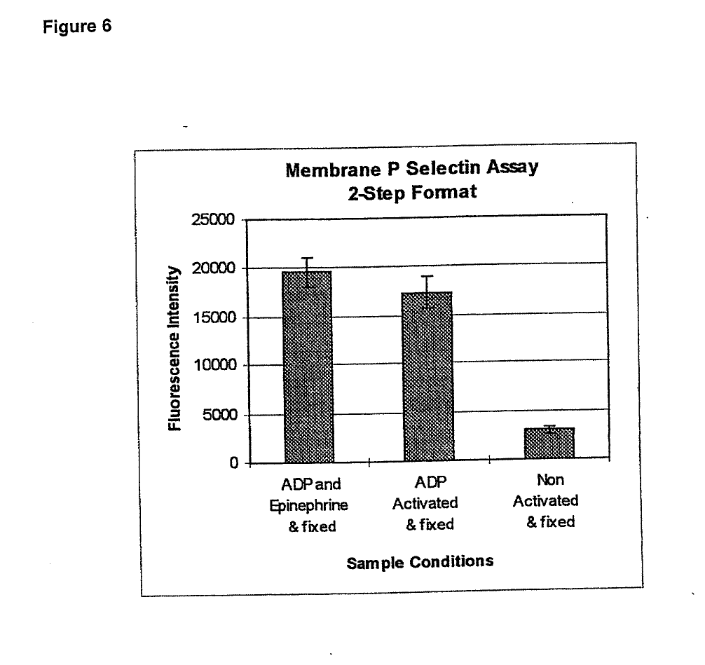

The data in table 4 as well as the graph in FIG. 6 illustrate how the incubation of whole blood with paramagnetic microparticles coated with anti-GP1b murine monoclonal antibodies followed by incubation with labeled anti-P-selectin rabbit polyclonal antibodies enables the detection, isolation and discrimination of platelets that express membrane P-selectin from those platelets that do not express membrane P-selectin.

TABLE 4
FITC Signal Detected vs. Sample Preparation Conditions

| Sample Preparation Conditions | FITC signal detected |
|---|---|
| ADP & Epinepherine agonist treated whole blood | 19550 |
| ADP agonist treated whole blood | 17349 |
| Non agonist treated whole blood | 2986 |

### EXAMPLE 7

### GPIIb/IIIa Assay

3.7 micron (μm) carboxyl-modified paramagnetic microparticles were coated with Human Fibrinogen which interacts and binds with the complexed form of the platelet membrane surface component glycoproteins GPIIb/GPIIIa (CD41/CD61). The coated particles were re-suspended in phosphate buffered saline (PBS) buffer at 0.5% w/v.

Whole blood was obtained from a healthy volunteer and collected into a 3.0 mL blood collection tube containing 0.3 μg D-phe-pro-arg-chloromethylketone (PPACK, Dihydrochloride) and 2.1 mL Hanks' Balanced Salt supplemented with 10 mM HEPES, pH 7.4. An aliquot of this whole blood preparation was stimulated with 10 μM adenosine 5′-diphosphate (ADP) for two minutes prior to being tested in the assay. Another aliquot was stimulated with 2 μM adenosine 5′-diphosphate (ADP) for two minutes prior to being tested in the assay, and a final aliquot was not treated with ADP.

To a set of 5 μL, 10 μL, and 20 μL of each of the above three sample preparations 20 μL of FITC-labeled anti-IIb/IIIa (anti-CD41a) mouse monoclonal antibody (at 20 μg/mL) was added. The mixture was incubated for 10 minutes at room temperate at which point 50 μL of 0.5% (w/v) paramagnetic microparticles (3.7 micron (μm)) coated with Fibrinogen was added. The mixtures were incubated at room temperature for an additional 10 minutes. The microparticles were separated magnetically and the supernatant was removed. The microparticles were washed by re-suspension in 200 μL of PBS supplemented with 1% bovine serum albumin (BSA), followed by magnetic separation of the microparticles and removal of the supernatant. This sequence was repeated three times and the microparticles were re-suspended in 0.1% SDS at pH 11. The microparticles were separated magnetically and the supernatants were transferred to a 96 well plate and examined for fluorescence intensity.

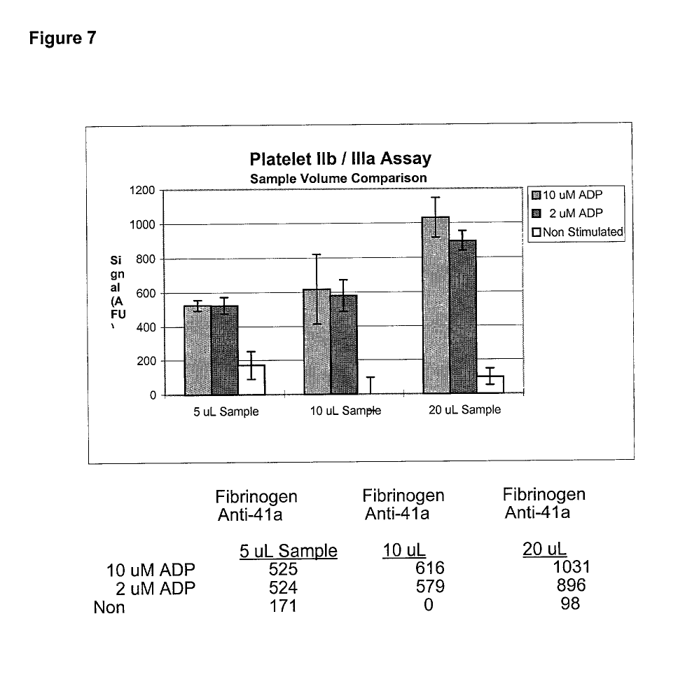

The data in table 5, as well as the graph in FIG. 7, illustrate how the incubation of whole blood with both paramagnetic microparticles coated with fibrinogen and a labeled anti-GPIIb/IIIa monoclonal antibody enables the detection and discrimination of whole blood samples containing platelets that are expressing membrane-bound GPIIa/IIIb from those that are not expressing membrane-bound GPIIa/IIIb.

TABLE 5
FITC Signal Detected vs. Sample Volume

| | 5 μL | 10 μL | 20 μL |
|---|---|---|---|
| 10 uM ADP agonist treated whole blood aliquot | 525 | 616 | 1031 |
| 2 uM ADP agonist treated whole blood aliquot | 524 | 579 | 896 |
| Non agonist treated whole blood aliquot | 171 | 0 | 98 |

### EXAMPLE 8

### Membrane P-Selectin Assay

Whole blood was obtained from a healthy volunteer and collected into a 3.0 mL blood collection tube containing 0.3 μg D-phe-pro-arg-chloromethylketone (PPACK, Dihydrochloride) and 2.1 mL Hanks' Balanced Salt supplemented with 10 mM HEPES, pH 7.4. An aliquot of this whole blood preparation was stimulated with 10 μM adenosine 5′-diphosphate (ADP) for two minutes prior to being tested in the assay. Another aliquot was stimulated with 2 μM adenosine 5′-diphosphate (ADP) for two minutes prior to being tested in the assay, and a final aliquot was not treated with ADP.

To a set of 10 μL and 20 μL of each of the above three sample preparations 20 μL of FITC-labeled anti P-Selectin (anti-CD62P) rabbit polyclonal antibody (at 20 μg/mL) was added. The mixture was incubated for 10 minutes at room temperate at which point 50 μL of 0.5% (w/v) paramagnetic microparticles (3.7 micron (μm)) coated with anti-glycoprotein GP1b (CD42b) murine monoclonal antibody (Mab) was added. The mixtures were incubated at room temperature for an additional 10 minutes. The microparticles were separated magnetically and the supernatant was removed. The microparticles were washed by re-suspension in 200 μL of PBS supplemented with 1% bovine serum albumin (BSA) followed by magnetic separation of the microparticles and removal of the supernatant. This sequence was repeated three times and the microparticles were re-suspended in 0.1% SDS at pH 11. The microparticles were separated magnetically, and the supernatants were transferred to a 96 well plate and examined for fluorescence intensity.

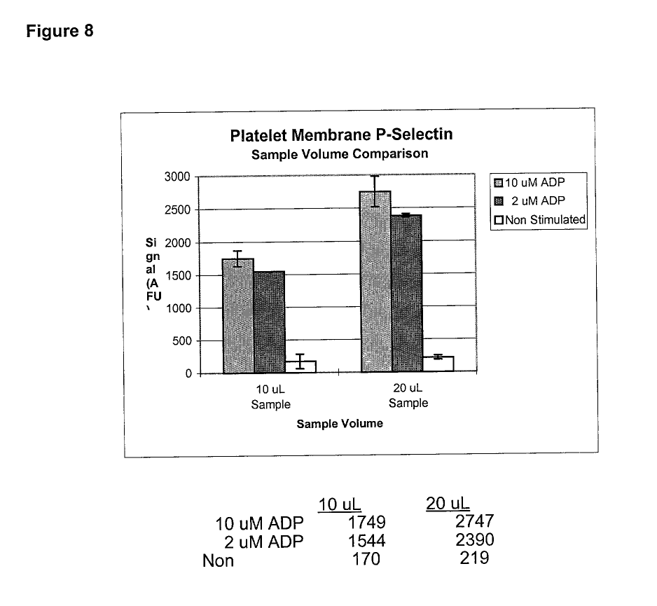

The data in table 6, as well as the graph in FIG. 8, illustrate how the incubation of whole blood with both paramagnetic microparticles coated with an anti-GP1b murine monoclonal antibody and a labeled anti P-selectin rabbit polyclonal antibody, enables the detection and discrimination of whole blood samples containing platelets that are expressing membrane-bound P-Selectin from those that are not expressing membrane-bound P-Selectin.

TABLE 3
FITC Signal Detected vs. Sample Volume

| | 10 μL | 20 μL |
|---|---|---|
| 10 μM ADP agonist treated whole blood aliquot | 1749 | 2747 |
| 2 μM ADP agonist treated whole blood aliquot | 1544 | 2390 |
| Non agonist treated whole blood aliquot | 170 | 219 |

The above set-out examples serve to illustrate the invention and are not intended to limit it in spirit or scope. Those of skill in the art will appreciate that numerous deviations from the examples described above can be made or performed without exceeding the scope of the invention. All patents and patent applications (published or unpublished) as well as other scientific and technical literature referred to herein are expressly incorporated herein by reference to the extent that they are not contradictory.

What is claimed is:

1. A method for analyzing a biological sample, wherein the method comprises the steps of:
(a) combining the biological sample, a coated solid phase and one or more marker-specific, labeled compounds to form a reaction mixture, and
(b) analyzing the mixture for the presence and/or concentration of the marker.

2. The method of claim 1, wherein the solid phase is separated from the mixture prior to analysis.

3. The method of claim 1, wherein the biological sample comprises undiluted or diluted whole blood.

4. The method of claim 1, wherein the biological sample comprises undiluted or diluted blood plasma.

5. The method of claim 1, wherein the biological sample comprises a undiluted or diluted fraction of fractionated whole blood.

6. The method of claim 1, wherein the solid phase comprises paramagnetic particles.

7. The method of claim 6, wherein the solid phase is separated from the biological sample with a magnet prior to analysis.

8. The method of claim 6, wherein the particles are coated with antibodies or fragments of antibodies with intact complementary determining regions.

9. The method of claim 8, wherein the antibodies or fragments of antibodies are monoclonal or polyclonal.

10. The method of claim 1, wherein the solid phase is coated with one or more substances selected from the group consisting of polyclonal antibodies, monoclonal antibodies, receptors, ligands, proteins, peptides, cytokines, chemokines, small molecules and fragments of any of the preceding.

11. The method of claim 1, wherein the marker-specific, labeled compounds comprise antibodies.

12. The method of claim 1, wherein the label comprises a fluorophore.

13. The method of claim 1, wherein the marker is selected from the group consisting of: CD42c (GP1b-beta)-25 kD disulfide bonded to alpha subunit; CD42d (GPV); CD41 (GPIIb also known as alpha IIB integrin); CD61 (GPIIIa)-beta 3 subunit of GPIIb/IIIa complex (alpha 2b, beta 3); CD41/CD61 (GPIIb/IIIa complex)—receptor for fibrinogen, fibronectin, von Willebrand factor, and other adhesion proteins containing the Arg-Gly-Asp motif; CD36 (GPIV)-platelets/monocytes; CD49b (VLA-2)-platelets/monocytes; CD51 (alpha V, beta 3)-vitronectin receptor; CD62p (P-selectin)-platelets; selectin)-platelets; CD107a (LAMP-2)-lysosomal protein translocated to cell surface after activation and CD41a (GPIIb/IIIa)—intact IIb/IIIa complex; fibrinogen, von Willebrand factor, fibronectin and vitronectin receptor.

14. The method of claim 1, wherein said solid phase is coated with a substance specific for a marker selected from the group consisting of: CD42c (GP1b-beta)-25 kD disulfide bonded to alpha subunit; CD42d (GPV); CD41 (GPIIb also known as alpha IIB integrin); CD61 (GPIIIa)-beta 3 subunit of GPIIb/IIIa complex (alpha 2b, beta 3); CD41/CD61 (GPIIb/IIIa complex)—receptor for fibrinogen, fibronectin, von Willebrand factor, and other adhesion proteins containing the Arg-Gly-Asp motif; CD36 (GPIV) platelets/monocytes; CD49b (VLA-2)-platelets/monocytes; CD51 (alpha V, beta 3)-vitronectin receptor; CD62p (P-selectin)-platelets; CD107a (LAMP-2)-lysosomal protein translocated to cell surface after activation and CD41a (GPIIb/IIIa)—intact IIb/IIIa complex; fibrinogen, von Willebrand factor, fibronectin and vitronectin receptor.

15. The method of claim 1, wherein said marker-specific, labeled compound is specific for a marker selected from the group consisting of: CD42c (GP1b-beta)-25 kD disulfide bonded to alpha subunit; CD42d (GPV); CD41 (GPIIb also known as alpha IIB integrin); CD61 (GPIIIa)-beta 3 subunit of GPIIb/IIIa complex (alpha 2b, beta 3); CD41/CD61 (GPIIb/IIIa complex)—receptor for fibrinogen, fibronectin, von Willebrand factor, and other adhesion proteins containing the Arg-Gly-Asp motif; CD36 (GPIV)-platelets/monocytes; CD49b (VLA-2)-platelets/monocytes; CD51 (alpha V, beta 3)-vitronectin receptor; CD62p (P-selectin)-platelets; CD107a (LAMP-2)-lysosomal protein translocated to cell surface after activation and CD41a (GPIIb/IIIa)—intact IIb/IIIa complex; fibrinogen, von Willebrand factor, fibronectin and vitronectin receptor.

16. The method of claim 1, wherein a buffer is included in the reaction mixture.

17. The method of claim 9, wherein the buffer comprises Hepes buffer, Hank's balanced salts and PPACK.

18. The method of claim 2, wherein the biological sample comprises undiluted or diluted whole blood; the solid phase comprises paramagnetic particles; the paramagnetic particles are coated with one or more polyclonal antibodies, one or more monoclonal antibodies, and/or fibrinogen; the marker-specific, labeled compound comprises one or more fluorophore-labeled polyclonal and/or monoclonal antibodies that recognize activated and/or unactivated platelets; the analysis measures the presence, absence and/or concentration of activated and/or unactivated platelets; and the solid phase is separated magnetically from the biological sample.

19. Cells separated from whole blood according to the method of claim 18, wherein the separated cells comprise physiologically activated platelets, unactivated platelets and/or platelet-derived microparticles.

20. A method for analyzing the biological sample and separated cells of claim 18 in order to assess the relative risk of acute coronary syndrome(s) in the patient or subject from whom the biological sample was obtained.
